(19) United States
(12) Patent Application Publication
(10) Pub. No.: US 2003/0096017 A1
(43) Pub. Date: May 22, 2003

(54) Anti-microbial powder coatings

(76) Inventors: Owen H. Decker, Houston, TX (US); Michael A. Sparks, Houston, TX (US); Jeffrey R. Hagerlin, Houston, TX (US); Frederick L. Cummings, Richmond, TX (US); Peter Gottschling, Pierrefonds (CA)

Correspondence Address: TROP PRUNER & HU, PC, 8554 KATY FREEWAY, SUITE 100, HOUSTON, TX, 77024, US

(21) Appl. No.: 10/185,545

(22) Filed: Jun. 28, 2002

Related U.S. Application Data
(63) Continuation-in-part of application No. 09/713,882, filed on Nov. 16, 2000, now Pat. No. 6,432,416.
Application No. 09/713,882 is a continuation-in-part of application No. 09/624,155, filed on Jul. 24, 2000, now abandoned.
Application No. 09/624,155 is a continuation of application No. 09/165,839, filed on Oct. 2, 1998, now Pat. No. 6,093,407.
(60) Provisional application No. 60/061,099, filed on Oct. 3, 1997.

(51) Int. Cl. A61K 9/50; A61K 33/24; A61K 33/38

(52) U.S. Cl. 424/617; 424/618; 424/501

(57) ABSTRACT

Improved powder coatings exhibit enhanced resistance to bacterial and fungal attack, while possessing excellent toughness, appearance, corrosion resistance, durability, processability, and ease of application. The coating is comprised of anti-microbial agents that are homogeneously dispersed within the powder or impact fused to powder particles or both. An article may be coated with a thermoset thermoplastic, or radiation curable powder, which may be applied by electrostatic spray or fluidized bed or by thermal or flame spray.

This application is a continuation-in-part of application Ser. No. 09/713,882 filed on Nov. 16, 2000, which is a continuation-in-part of application Ser. No. 09/624,155 filed on Jul. 24, 2000, now abandoned, which is a continuation of application Ser. No. 09/165,839 filed on Oct. 2, 1998, that was issued as U.S. Pat. No. 6,093,407 on Jul. 25, 2000, that was based on provisional application Serial No. 60/061,099, filed on Oct. 3, 1997.

### BACKGROUND

This invention relates generally to powder coatings and particularly to anti-microbial powder coatings.

Public concern about the health hazards arising from microorganisms such as bacteria, fungi, viruses and the like is high. Many people are concerned that contact with objects in public facilities may result in illness. Also, it is desirable to prevent biological defacement of object surfaces due to the growth of microorganisms.

Thus, a number of efforts have been undertaken to produce objects with the ability to kill or inhibit the growth or reproduction of microorganisms, which is termed “anti-microbial activity” herein. For example, plastic materials with anti-microbial activity are known. The resulting plastic products then exhibit some degree of anti-microbial activity.

For example, some toys for young children include anti-microbial agents (i.e., agents with anti-microbial activity) within a plastic matrix. These anti-microbial agents, which are believed to be safe, are believed to inhibit the growth of various microorganisms. Anti-microbial agents in the final coatings including paint and powder coatings are known. However, none of the existing techniques in powder coatings have gained substantial acceptance.

Therefore, there is a continuing need for improved coatings and particularly for improved powder coatings that exhibit anti-microbial activity when applied to substrates.

### BRIEF DESCRIPTION OF THE DRAWING

FIG. 1 is a diagrammatic depiction of a process for making a powder coating.

### DETAILED DESCRIPTION

A stable anti-microbial powder coating composition may coat a product that may be exposed to bacteria and fungal spores. In one embodiment, the powder coating may be made by a process that produces a homogeneous distribution of anti-microbial agents that may promote consistent and efficient anti-microbial activity. Once coated with the anti-microbial powder coating, a substrate may be protected from physical abuse by the film's physical properties and durability and from degradation due to attack by microorganisms and also potentially protecting the user from various microorganisms.

The powder coating formulation may be applied to the substrate so that bacterial or fungal contact with the coating either kills them or at least inhibits their growth. For example, in some embodiments, anti-microbial activity with respect to Staphylococcus aureus, Escherichia coli, Bacillus subtillus, Streptococcus faecadis, Salmonella typhinurium, Pseudomonas aeruginosa, and other Gram positive and Gram negative bacteria may be achieved. Powder coating formulations, in some embodiments, may also inhibit the growth of certain higher organisms like algae, fungi, filamentous fungi (Aspergillus, Aureobasidium, Botrytis, Ceratostomella, Cuvularia, Fusarium and Penicillium species), yeast and also, some viruses.

Potential applications for these improved powder coatings may include, for example, food preparation areas, restrooms, hospitals, garbage disposals, stockyard areas, animal feed troughs, schools, kitchens, swimming pool areas, dishwashers, automobile fixtures, public access fixtures, public seating, public transportation fixtures, toys, and other industrial, agricultural, commercial or consumer products.

The resin may be one or more of the thermosetting and/or thermoplastic resins including those based on epoxy, polyester, acrylic, polysiloxane and/or polyurethane resins. The coating may also include from about 0.1 percent to about 12 percent by weight of the total composition of one or more liquid or solid anti-microbial agents.

Examples of thermoplastic or thermosetting coatings that may be used include but are not limited to epoxies, saturated and unsaturated polyesters, carboxylic acid-functional polyesters, hydroxyl-functional polyesters, epoxy/polyester hybrids, acrylics, epoxy/acrylic hybrids, glycidyl-functional acrylics, polyester-urethanes, acrylic urethanes and siloxanes. Thermoplastic powder coatings that may be useful include but are not limited to nylon, polyvinyl chloride (PVC), polyethylene, polyethylene terephthalate (PET), polybutylene terephthalate (PBT), and polypropylene as examples. These powder coatings may be cured or fused by thermal or photochemical methods.

The anti-microbial agents include but are not limited to phthalimides, acetamides, phthalonitriles, hydroxy benzoates, isothiazolinones, nitropropane diols, carbamates, methyl ureas, benzimidazoles, salicylanilides, mercury acetates, organozinc compounds, metals such as silver, copper and zinc, and ions of such metals.

Among the liquid anti-microbial agents that are suitable in certain applications, a preferred anti-microbial agent is dibromocyanoacetamide (for example, Amerstat® 300 made by Drew Industrial Division of Ashland Chemicals, Boonton, N.J. 07005).

In addition, solid anti-microbial agents that are preferred include 2-bromo-2-nitropropane-1,3-diol (for example, Canguard® 409 made by Angus Chemical Co., Buffalo Grove, Ill. 60089) and 3,5-dimethyltetrahydro-1,3,5-2H-thiazine-2-thione (for example, Nuosept® made by Creanova, Inc., Piscataway, N.J. 08855 or Troysan® 142 made by Troy Chemical Corp., West Hanover, N.J. 07936).

Other solid anti-microbial agents include N-(trichloromethyl)-thiophthalimide (for example, Fungitrol® 11 made by Creanova, Inc.), butyl-p-hydroxy-benzoate (for example, Butyl Parabens® made by International Sourcing Inc., Upper Saddle River, N.J. 07458), diiodomethyl-p-tolysulfone (for example, Amical® WP made by Angus Chemical Co.), and tetrachloroisophthalonitrile (for example, Nuocide® 960 made by Creanova, Inc.).

Metals such as silver, copper and zinc and their metal ions also have anti-microbial properties. Silver ions have widespread effect as an anti-microbial agent. For example, silver ions may be effective against bacteria such as Escherichia coli and Salmonella typhimurium, and mold such as Asperigillus niger.

Sources of silver for anti-microbial use include metallic silver, silver salts and organic compounds that contain silver. Silver salts may include for example, silver carbonate, silver sulfate, silver nitrate, silver acetate, silver benzoate, silver chloride, silver fluoride, silver iodate, silver iodide, silver lactate, silver nitrate, silver oxide and silver phosphates. Organic compounds containing silver may include for example, silver acetylacetonate, silver neodecanoate and silver ethylenediaminetetraacetate in all its various salts.

Silver containing zeolites (for example, AJ10D containing 2.5% silver as Ag(I), and AK10D containing 5.0% silver as Ag(I), both made by AgION™ Tech. L.L.C., Wakefield, Mass. 01880) are of particular use. Zeolites are useful because when carried in a polymer matrix they may provide silver ions at a rate and concentration that is effective at killing and inhibiting microorganisms without harming higher organisms.

Silver containing zirconium phosphate (for example, AlphaSan RC 5000 containing 3.8% silver provided by Milliken Chemical, Spartanburg, S.C. 29304) is also particularly useful. In general zirconium phosphates act as ion exchangers. However, AlphaSan RC 5000 is a synthetic inorganic polymer that has equally spaced cavities containing silver, wherein the silver provides the anti-microbial effects. Silver zirconium phosphates are typically incorporated into powder coatings between 0.1 and 10 percent by weight and particularly 0.5 to 5 percent by weight of the total powder coating formulation.

The powder coating may be sprayed electrostatically onto a metal or nonmetal substrate. In this method, the substrate may be grounded. Charged particles of the powder coating are sprayed onto the substrate until a desired thickness is achieved. Other methods, such as fluidized bed coating methods or thermal or flame spraying, may also be used.

After the deposition is complete, the coated substrate is heated. For example, an electrical or gas fired oven may be used to cure or fuse the coating at temperatures in the range of 80° C. to 270° C. The curing time may be about five to twenty minutes for most substrates, but may vary from less than a minute to greater than one hour depending on the type of coating, the substrate, and the curing system.

Advantageously, visible bubbling in the coating film after the curing process should be avoided. The presence of bubbles may indicate that some of the biocide may have been volatilized during the curing process. Advantageous anti-microbial agents should not produce visible bubbles indicative of volatilizing of the active element.

In addition to thermal curing, some powder coating formulations may be cured by radiation such as ultraviolet (UV) or electron beam radiation. That is, instead of heat, a source of radiation initiates the cure process. Radiation curing has several advantages over thermal curing. For example, radiation curing may be used to cure coatings on heat sensitive substrates such as wood or plastic. Moreover, radiation curing generally has shorter cure times and is considered environmentally friendly. A detailed discussion of radiation curing and specifically UV curing may be found in U.S. Patents number 5,877,231 and 5,935,661, which are incorporated herein by reference. A general description of UV radiation curing is described as follows.

There are three general types of UV cured powder coating systems; photolytically initiated free radical systems, photolytically initiated cationic systems, and dual cure systems that utilize a photolytic system and a thermal system. All three systems incorporate at least one photoinitiator into the powder coating formulation. Typically, the photoinitiator absorbs a source of radiation, such as UV light to generate either free radicals or cations, depending on the photoinitiator being used. The resultant free radicals or cations initiate the polymerization and/or crosslinking reactions that constitute the UV cure process. In dual cure systems, a thermal initiator may also be added to the powder coating formulation to initiate curing by thermal means. Radicals or cations that are produced by thermal means may also be used in the absence of photogenerated radicals or cations to initiate crosslinking reactions.

There are many commercially available photoinitiators including free radical initiators such as IRGACURE® 2959 manufactured by Ciba® Specialty Chemicals, Inc., Tarrytown, N.Y. 10591. Other free radical photoinitiators include benzoin and its derivatives such as isobutyl benzoin ether and benzyl dimethly ketal, acyl phosphines such as 2,4,6-trimethylbenzoyl diphenylphosphine oxide, aryl ketones such as 1-hydroxycyclohexyl phenyl ketone, and benzophenone, ketocoumarin and anthracene or its derivatives.

Cationic photoinitiators include, but are not limited to, diaryliodonium salts, triarylsulfonium salts such as triphenyl sulphonium hexafluorophosphate, triphenyl sulphonium tertafluoroborate and the like.

Suitable free radical thermal initiators include organic peroxides, such as benzoyl peroxide; diacyl peroxides, such as 2-4-diclorobenzoyl peroxide, diisononanoyl peroxide, decanoyl peroxide, lauroyl peroxide, succinic acid peroxide, acetyl peroxide, and diisobutyryl peroxide; acetyl alkylsulfonyl peroxides, such as acetyl cyclohexylsulfonyl peroxide; dialkyl peroxydicarbonates, such as di(n-propyl)peroxy dicarbonate, di(sec-butyl)peroxy dicarbonate, di(2-ethylhexyl)peroxy dicarbonate, diisopropylperoxy dicarbonate, and dicyclohexylperoxy dicarbonate; peroxy esters, such as α-cumylperoxy neodecanoate, α-cumylperoxy pivalate, t-amylperoxy neodecanoate, t-butylperoxy neodecanoate, t-amylperoxy pivalate, t-butylperoxy pivalate, 2,5-dimethyl-2,5-di(2-ethylhexanoylperoxy)hexane, t-amylperoxy-2-ethyl hexanoate, t-butylperoxy-2-ethyl hexanoate, and t-butylperoxy isobutyrate; azobis (alkyl nitrile) peroxy compounds, such as 2,2′-azobis-(2,4-dimethylvaleronitrile), azobisisobutyronitrile, and 2,2′-azobis-(2-methylbutyronitrile); t-butyl-peroxymaleic acid, 1,1′-azobis-(1-cyclohexanecarbonitrile); peroxy ketals, such as 1,1-di(t-butylperoxy)-3,3,5-trimethylcyclohexane; peroxy esters, such as o,o′-t-butyl-o-isopropyl monoperoxy carbonate, 2,5-dimethyl-2,5-di(benzoylperoxy) carbonate, o,o′-t-butyl-o-(2-ethylhexyl)-monoperoxy carbonate, t-butylperoxy acetate, t-butylperoxy benzoate, di-t-butyldiperoxy azelate, and di-t-butyldiperoxy phthalate; dialkylperoxides, such as dicumyl peroxide, 2,5-dimethyl-2,5-di(t-butylperoxy)hexane, t-butyl cumyl peroxide, di-t-butyl peroxide, and 2,5-dimethyl,2,5-di(t-butylperoxy)hexyne-3; hydroperoxides, such as 2,5-dihydroperoxy-2,5-dimethyl hexane, cumene hydroperoxide, t-butyl hydroperoxide and t-amyl hydroperoxide, ketone peroxides; such as n-butyl-4,4-bis-(t-butylperoxy)valerate, 1,1-di(t-butylperoxy)-3,3,5-trimethyl cyclohexane, 1,1′-di-t-amyl-peroxy cyclohexane, 2,2-di(t-butylperoxy) butane, ethyl-3,3-di(t-butylperoxy)butyrate, and blend of t-butyl peroctoate, and 1,1-di(t-butylperoxy)cyclohexane; and diazo compounds, such as 1,1′-azobis(cyclohexanecarbonitrile)peroxide.

Additional thermal and photoinitiators are disclosed in U.S. Pat. Nos. 5,922,473; U.S. 6,005,017; and U.S. 6,017,640, all of which are incorporated herein by reference. Other components of UV curable powder coatings may include resins, crosslinkers, and other common additives such as flow agents, performance enhances, fillers and pigments.

Typical resins found in free radical and dual cure systems include ethylenically unsaturated resins. Especially common are unsaturated polyesters, vinyl ethers, acrylourethanes, acrylated epoxies and acrylated acrylics. Moreover, optional ethylenically unsaturated crosslinking agents may include one or more of the following functional group types: maleates, fumarates, acrylates, methacrylates, vinyl esters, vinyl ethers and styrenes. These moieties may be readily appended to or incorporated into a wide variety of organic and inorganic materials including polyols, polyamines, polycarboxylic acids, polyolefins, polyesters, polyurethanes, polyamides, polyacrylates and polysiloxanes.

Typical resins found in cationically cured systems include epoxies and vinyl ethers and, optionally, crosslinking agents with one or more of the following cationically polymerizable functional groups: epoxies (especially cycloaliphatic epoxies) and vinyl ethers.

These UV curable powder coating formulations may be made by the melt extrusion method, as described below. Moreover, anti-microbial agents such as silver zeolite may also be added to UV curable powder coating formulations either by blending during premixing in the melt extrusion process or by impact fusion, both of which are described below.

The UV powder coatings may be deposited on a substrate as previously described. However, after deposition and before curing, the coated substrate, may first be exposed to heat so that the powder will become molten and flow over the substrate. Infrared radiation or convection oven or both may provide heat. However, the heat used to cause the powder to flow is less than the heat needed for thermal curing. After exposure to heat, the substrate may be cured by exposure to radiation such as UV or electron beam radiation so that the powder coating will harden. Sources of UV radiation include mercury lamps, such as H lamps and gallium or iron-doped mercury lamps, all manufactured by Fusion UV Systems, Inc., Gaithersburg, Md. 20878-1357.

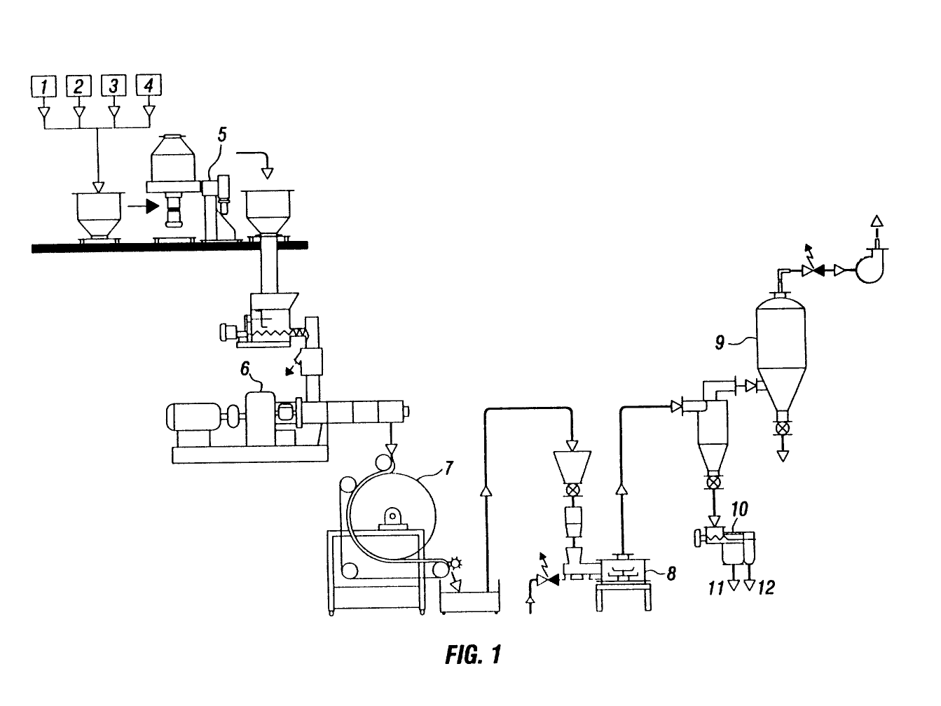

Powder coatings may be made by a melt extrusion method, as illustrated in FIG. 1. For example, a powder formulation includes more than one ingredient as represented by items 1-4. Fillers, extenders, flow additives, catalysts, hardeners, pigments and other additives may be blended together with the resin and the anti-microbial agent in a premixer 5. The mixture may then be fed into an extruder 6 and heated to a temperature high enough to melt and mix the constituents. A temperature in the range of 50° C. to 150° C. may be sufficient. The molten extrudate may be immediately cooled by chill rolls 7 to form solid sheets.

The solid sheets may be further broken down to suitably sized chips. These chips are then fed into a grinder 8 that reduces the chips to fine particles. For example, particles having a mean particle size of about 10 microns to 180 microns are satisfactory. The resulting powder advantageously has a glass transition temperature that is greater than the storage temperature. A dust filter 9, a sieve screen 10, and powder inspection station 11 and 12 may also be provided.

The anti-microbial agents may be uniformly dispersed in the resin formulation (including the curing agent) during the premix stage. This is advantageous because there is no requirement that the anti-microbial agents have a specific particle size or particle size distribution. The anti-microbial agents are chosen to survive the extrusion process and the subsequent curing process in sufficient concentration to exhibit an anti-microbial effect in the final coating. In addition, it is preferable that the anti-microbial agent does not adversely change any important property of the final coating such as color, coalescence or cure speed.

In some cases, the efficacy of a particular anti-microbial agent may be affected by the presence of another component in the powder coating formulation, such as species that decrease the anti-microbial agent's solubility, chelators or other substances that sequester anti-microbial agents and acids or bases. These species may reduce the anti-microbial agent's efficacy by inhibiting the agent's ability to migrate through the coating to the surface.

For example, anti-microbial metals, such as silver, may react with species such as acetylide, azide, bromide, chloride, cyanide, ferricyanide, ferrocyanide, iodide, oxide, phosphate, sulfide, thiocyante and silver free zeolites to form low solubility compounds. Thus, the use of these species should be minimized in coatings that utilize anti-microbial metals.

Namely, in silver-ion-based anti-microbial powder coatings, it is preferred to eliminate from the powder coating formulation any component that contributes a substantial quantity of ionic halides including bromide, chloride, iodide and fluoride or other silver binding species such as acetylide, azide, cyanide, ferricyanide, ferrocyanide, oxide, phosphate, sulfide, thiocyante and silver-free zeolites that are not already associated with the silver ion. Specifically, the contributing component should not contribute more than 300 parts per million (ppm) of the above-listed species. However, it is preferred that the contributing component contribute no more than 50 ppm and it is particularly preferred that the contributing component contribute no more than 10 ppm of the above-listed species that are not already associated with silver ions. These proportions are particularly important for components that contribute ionic halides that are not already associated with silver ions.

Polymer-bound chelators may also reduce the efficacy of anti-microbial metals, such as silver. For example, polycarboxylates, polyamines, polyethers (including crown ethers) and other species with metal chelating functional groups that may bind the metal thereby reducing the anti-microbial metal's ability to migrate through the powder coating.

Although under certain circumstances certain chemical species may reduce an anti-microbial metal's ability to migrate through a powder coating, under different circumstances these same species may serve as useful anti-microbial compounds. Generally, those species that have the ability to trap, capture or sequester an anti-microbial agent may also have the ability to carry the anti-microbial agent and release it at a controlled rate. For instance, silver carriers such as silver salts including silver phosphates and silver zeolites are desirable anti-microbial agents that exhibit controlled release rates of the anti-microbial metal. Nevertheless, when not in association with an anti-microbial metal, these species are deleterious and their presence in powder coatings should be minimized.

The ability of organic anti-microbial agents to migrate to the surface of powder coatings may be affected by chemical species known to sequester organic compounds, such as empty zeolites and cyclodextrins with a pore size larger than 5 angstroms. Moreover, the migration of basic anti-microbial agents may be reduced by the presence of acidic sites and the migration of acidic anti-microbial agents may be reduced by basic sites.

Solid anti-microbial agents including those that are metallic or metal-containing may be blended directly with the formulation components before extrusion. Alternatively, the particles of anti-microbial agents may be bonded to powder coating component particles after extrusion and grinding using impact fusion. In some instances, it may be desirable to blend the anti-microbial agent with the formulation components before extrusion and bond the same or a different anti-microbial agent to the powder coating particles after extrusion and grinding.

Impact fusion is a process wherein the anti-microbial particles and the powder or component particles fuse at points of impact. Impact fusion is also known in the art as fusion bonding or impact bonding. For example, when using impact fusion to bond anti-microbial agents to powder coating particles, the anti-microbial agent may be eliminated from the melt extrusion method's blended premix. Instead, the powder coating composition, without the anti-microbial agent, may be prepared and melt extruded, solidified, ground into a powder and scalped to yield the powder coating powder. After scalping, the anti-microbial agent may be mixed with the powder coating powder and blended with a high intensity mixer. During blending, the powder coating particles and the anti-microbial agents fuse to make a homogeneous mixture where the anti-microbial agent is substantially entirely fused to the surface of the powder particles.

With either blending and premixing the anti-microbial agent with the powder coating components or impact fusion of the anti-microbial agent to the powder coating powder particles, blending, mixing or impact fusing the anti-microbial particles with coating particles of the same particle size distribution is not necessary.

Liquid anti-microbial agents can be mixed readily with other components in the premix prior to extrusion. Liquid anti-microbial agents often are difficult to dry blend into a powder to a concentration that consistently, effectively protects against bacteria or fungi. Alternatively, liquid anti-microbial agents may be mixed initially with particles of a solid support material such as a silica, clay or other resins in a masterbatch. The dry mixture containing the liquid anti-microbial agent may then be mixed into a formulation of resin.

For example, the liquid anti-microbial agent may be mixed at room temperature using high shear into fumed silica yielding high concentrations of active ingredients. The resulting granular solid may then be treated as a solid anti-microbial agent. For example, concentrations of approximately 66 percent of active ingredients in fumed silica may be utilized.

Liquid and solid anti-microbial agents also may be incorporated within the powder coating particle by dissolving or mixing them and the other powder coating formulation components in a suitable solvent, e.g., organic liquids or supercritical fluids, and then removing the liquid in such a manner as to yield a powder or a solid product which can be processed into a powder.

A suitable powder coating material, which is utilized in examples one through four is Gold Bond III, a catalyzed epoxy powder coating sold by DuPont Powder Coatings, Inc., of Houston, Tex. Fillers and extenders, melt flow additives, dry flow additives, pigments and other additives may also be used to enhance specific physical properties, aesthetics, durability or other attributes.

Other powder coating materials, utilized in examples five and six, include urethane-cured polyesters compositions and triglycidylisocyanurate (TGIC)epoxy-cured polyesters compositions. Fillers, flow aids, degassing aids and pigments may also be used to enhance certain properties of the powder coating such at aesthetics and durability.

### EXAMPLE 1

A long-term anti-microbial activity test was carried out to determine if selected anti-microbial agents maintain their anti-microbial activity after being incorporated into powder coatings and cured.

Six anti-microbial agents were selected for experimentation. They are Fungitrol® 11, Amerstat® 300, Nuocide® 960, Nuosept® S, Propyl Parabens®, and Butyl Parabens®. For each powder coating formulation, one of the six anti-microbial agents was added at concentrations of 0.1 percent and 1 percent of the total resin weight.

Samples containing one of the six additives at the two concentrations in the coating matrix were prepared. The samples are coated on 2.54 cm. by 2.54 cm. by 0.08 cm. steel coupons. Both the front and the back of the coupons were coated with a given coating formulation. The edges were coated with a black silicone resin to prevent rusting of the coupon, which might interfere with the interpretation of the experimental results. Controls containing the coating formulation with no additive and controls containing only the black silicone resin used for edge coverage were included.

The target bacterial organisms were Pseudomonas aeruginosa, Escherichia coli, and Salmonella typhinurium. Five groups of samples were prepared. For each of the six additives, two panels with a coating thickness of 7 to 8 mils were cured with a normal schedule of 193° C. for 10 minutes. For each of the six additives, two panels with anti-microbial agent concentrations of 0.1 percent and 1 percent and with a coating thickness of 7 to 8 mils were cured with a normal schedule. Each of the following samples was prepared with an additive concentration of 1 percent of the resin by weight. For each of the six additives, two panels were prepared with a coating thickness of 3 to 4.5 mils and cured with a normal schedule.

The results were then rated on a scale of “0” (good performance) to “4” (poor performance) based on the number of colony-forming units observed. The growth ratings, which were averaged over the different samples, are based on the following numerical rating system:

0=No contamination (sterile).

1=Trace of contamination (1-9 colonies per “streak-inch”).

2=Light contamination (10-99 colonies per “streak-inch”).

3=Moderate contamination (greater than 100 colonies, but still distinguishable).

4=Heavy contamination (continuous smear of growth).

Resistance to fungal growth was tested generally in accordance with ASTM D5590-95. The organisms targeted were Aspergillus niger (ATCC 6275), Penicillium funiculosum (ATCC 11797), and Aureobasidium pullulans (ATCC 9348) in a mixed spore suspension. Samples were aseptically placed onto a modified malt agar plate and then each sample was inoculated. The following data was determined after four weeks:

| ADDITIVE | AVERAGE BACTERIAL COVERAGE |
|---|---|
| CONTROLS | 3.0 ± 1.7 |
| FUNGITROL ® 11 | 2.8 ± 1.8 |
| PROPYL PARABENS ® | 3.7 ± 0.5 |
| BUTYL PARABENS ® | 2.6 ± 1.3 |
| AMERSTAT ® 300 | 1.6 ± 1.5 |
| NUOCIDE ® 960 | 3.0 ± 1.3 |
| NUOSEPT ® S | 3.6 ± 0.5 |

The Amerstat® 300 showed significantly improved bacterial coverage compared to the other additives and the control. The effect of decreasing the additive concentration was minimal. Decreasing the coating thickness had very little effect on the anti-microbial activity of the coating.

Among the tested additives, Propyl Parabens® and Nuosept® S did not appear to improve the activity relative to the control and thus it was concluded that little or no effect on the long-term anti-microbial properties given the chosen resin matrix.

The same samples were also exposed to fungus spores for a period of four weeks. Results of the study showed that several of the coatings showed no growth of fungi on their surface after four weeks of exposure. At a concentration of 1 percent, powder coatings made with the Butyl and Propyl Parabens®, and Nuocide® 960 were free of visible fungal growth. Fungitrol® 11 and Amerstat® 300 had a very small amount of fungal growth. The Nuosept® S did not show conclusive fungal resistance.

An additional study was then undertaken using AATCC Test Method 30-1993, Part III. In this test, a control, Fungitrol® 11, Amerstat® 300, Nuocide® 960, Nuosept® S, Propyl Parabens® and Butyl Parabens® formulations were applied to steel coupons, as described previously. The samples were placed in sterile Petri dishes with Seboraud Dextrose Agar, inoculated with Aspergillus niger, (AATCC 6275), and incubated at 28° C. for three weeks. The fungus was placed on top of the coating as well as on the agar.

At the end of the three-week test period, only the control showed biological activity. When examined visibly and by microscope at one, two and three weeks, the control showed visible macroscopic fungal growth on its surface. The other formulations' surfaces did not have macroscopic or microscopic growth. Macroscopic growth was visible on the agar surfaces. However, it stopped at the coating edge. Since no zones of inhibition in the agar were visible, the anti-microbial agent is believed not to have leached out during exposure.

### EXAMPLE 2

In order to determine how fast the anti-microbial agents are able to work, shorter-term tests were also conducted. In many applications, it is desirable that the anti-microbial agent operates quickly.

Steel coupons were coated with the Fungitrol® 11, Butyl Parabens®, and Amerstat® 300 anti-microbial formulations, at 0.1 percent and 1 percent, and exposed as per ASTM D 5588-94 to a mixture containing the bacteria Pseudomonas aeruginosa, Staphylococcus aureus, and Escherichia coli. After the coupons were washed with a 70 percent ethanol/water solution, they were placed in a sterile Petri dish, inoculated, and incubated at 32° C. for the duration of the test.

At appropriate intervals, each sample was checked for the presence of viable microorganisms by streaking each sample with a sterile cotton swab, then streaking the swab onto Tryptic Soy Agar. The plates were incubated for 48 hours at 32° C. The absence of microbial growth along the streak indicated that the corresponding sample did not contain viable microbial cells. The presence of microbial growth would indicate non-sterility, i.e., the sample contained viable microbial contamination.

The samples were examined for low levels of bacterial contamination by transferring an aliquot with a sterile cotton swab to a Tryptic Soy Broth in culture tubes. The tubes were incubated for 24 hours at 32° C., streaked onto Tryptic Soy Agar plates and the plates were incubated for 24 to 48 hours at 32° C.

Heavy bacterial growth was detected initially and after 4 hours for all samples; however, after 24 hours of exposure, differentiation in growth was visible among the samples. After 72 hours of incubation, the Butyl Parabens®-coated samples were free of bacterial growth and were actually sterile. The control showed low to heavy growth. The Amerstat® 300 and Fungitrol® 11 did not show conclusive results.

Next, steel coupons were coated with the anti-microbial formulations listed above and exposed per ASTM D 5588-94 to a mixture containing the fungus spores of Aspergillus niger, Penicillium funiculosum, and Aerobasidium pullulans. After the coupons were washed with a 70 percent ethanol/water solution, they were placed in a sterile Petri dish, inoculated, and incubated at 28° C. for the duration of the test. At appropriate intervals, each sample was checked for the presence of viable microorganisms by streaking each sample with a sterile cotton swab, then streaking the swab onto Potato Dextrose Agar (adjusted to pH 3.5 for fungi). These plates were also incubated at 28° C.

The absence of microbial growth along the streak indicated that the corresponding sample did not contain viable microbial cells. The presence of microbial growth would indicate non-sterility, i.e., the sample contained viable microbial contamination.

Heavy fungal growth was detected initially and after 4 hours for all samples. However, once again, at 24 hours of exposure, differentiation among the samples was observed. After 72 hours of incubation, Fungitrol® 11- and Butyl Parabens®-coated samples were free of (or showed very low levels of) bacterial growth.

### EXAMPLE 3

Using AATCC Test Method 147 (Nutrient Broth, incubated at 37° C. for 18 to 24 hours), another test of very short term efficacy was undertaken. Cured powder coating formulations containing (0.1 percent and 1 percent) Fungitrol® 11, Amerstat® 300, Nuocide® 960, Nuosept® S, Propyl Parabens®, and Butyl Parabens® were exposed to a concentration of (inoculated) Staphylococcus aureus, Escherichia coli, and Salmonella choleraesuis for an exposure period of 18 to 24 hours. None of the formulations were effective in significantly killing the microorganisms over the short test cycle.

### EXAMPLE 4

The next experiments were conducted, according to the procedure of Example 3, to evaluate the effect of higher anti-microbial concentration on short-term anti-microbial activity. Coating powders containing 2 percent Arnerstat® 300, 4 percent Troysan® 174P, 5 percent Canguard® 409, 3 percent Irgasan® DP 400, 5 percent Amical® WP, 5 percent Nuosept® S, 10 percent Nuosept® S, 5 percent Nuocide® PCMC, and 10 percent Nuocide® PCMC were used in the next experiment.

Each formulation was loaded with anti-microbial agent until the powder became unstable. For example, if the powder sintered or cured too quickly, the concentration was reduced.

Significant zones of inhibition were achieved by the powder coatings containing 5 percent Canguard® 409, (bronopol), 3 percent Irgasan® DP 400 (triclosan, 5-chloro-2-(2,4 dichloro-phenoxy) phenol and 5 percent and 10 percent Nuosept® S. The bronopol (2-bromo-2-nitropropane-1,3-diol) formulation performed better than the triclosan formulation in inhibiting the growth of Escherichia coli and Salmonella choleraesuis.

The Nuosept® S performed as well as or better than the triclosan formulation in inhibiting the growth of Escherichia coli and Salmonella choleraesuis.

Thus, one preferred anti-microbial composition includes a mixture of anti-microbial agents that have short-term efficacy with agents having long-term efficacy. One preferred mixture includes 5 percent Nuosept® S and 0.1 percent Amerstat® 300 in a powder coating formulation.

### EXAMPLE 5

Experiments were performed to determine the concentration and anti-microbial effects of silver when a silver zeolite is incorporated into a powder coating composition. Silver zeolite was obtained from AgION™ Technologies, L.L.C., Wakefield, Mass. 01880. The zeolite was homogeneously distributed into powder coating mixtures during the premix as described previously. The resultant compositions were then melt-extruded, solidified between chilled rolls, and broken up and ground into a powder. The powders were scalped to remove particles larger than 180 microns.

Two types of powder coatings were prepared for these experiments. One powder coating type was a urethane-cured hydroxyl-functional polyester coating containing 100 parts of a hydroxyl resin (Ruco 102 HYD made by Ruco Polymer Corp., Hicksville, N.Y. 11801) and either 22.1 parts of a caprolactam-blocked isocyanate curing agent (Alcure 4400 made by McWhorter Tech. Inc., Ennis, Tex. 75119) or 27.7 parts of a uretidione-blocked isocyanate curing agent (Alcure 4147 made by McWhorter Tech. Inc.). See Table 1.

The other powder coating type was a triglycidylisocyanurate (TGIC)-cured carboxyl-functional polyester coating containing 100 parts of a carboxyl polyester resin (Uralac P-2400 made by DSM Resins US Inc., Augusta, Ga. 30903) and 7.5 parts of a TGIC curing agent (Araldite PT-810 made by Vantico Inc., Los Angeles, Calif. 90023). See Table 2.

Flow aids, fillers, degassing aids and pigments were also utilized in both types of powder coating compositions as indicated in Tables 1 and 2.

In the urethane-cured polyester composition, the silver zeolite AJ10D, containing 2.5% silver as Ag (I), was added so that its final concentration was one or three percent by weight of the total composition, as indicated in Table 1. In the TGIC-cured polyester composition, the same silver zeolite was added so that its final concentration was one, four or ten percent by weight of the total composition, as indicated in Table 2.

Both types of powder coating compositions were applied to grounded aluminum panels that were 0.020 inches thick. The powder coating thickness was from 1.8 to 2.5 mils. The powder coated panels were baked for 10 minutes at 400° F. and then evaluated for the concentration of silver in the coating, the concentration of surface silver and anti-microbial activity of the coating.

The total concentration of silver for each aluminum panel was determined from the amount of silver contained in the zeolite and the amount of zeolite added to each powder coating compositions. For example, the AJ10D zeolite has 2.5% Ag. If the zeolite made up 1% of the total powder coating composition, then the total concentration of silver in the composition would be 0.025%.

To determine surface silver concentration, the aluminum panels were trimmed to 2 inches by 2 inches and soaked for 24 hours in 25 mls. of 0.8M NaNO₃ solution. Aliquots of the solution were taken and tested for the concentration of silver ion by Graphite Furnace Atomic Absorption.

To determine silver's anti-microbial activity, 2 inch by 2 inch coated aluminum panels were inoculated with suspensions of Escherichia coli and incubated at 37° C. for 24 hours. The panels were then rinsed and the rinsate was serially diluted. The serial dilutions were applied to agar medium and further incubated to determine the percentage of bacterial growth.

Referring to Table 1, aluminum test panels coated with urethane-cured polyester compositions contained either 0, 1, or 3 percent of the total powder coating composition of silver zeolite, as shown in test panels 1 through 6. All panels were based on a hydroxyl polyester resin. Panels 1 through 5 were cured with a caprolactam-blocked isocyanate curing agent. Panel 6 was cured with a uretidione-blocked isocyanate curing agent. Panels 4 and 5 also contained pigment and filler. Flow aids and degassing aids were also present in the powder coating composition.

Still referring to Table 1, test panel 1, a control panel, had 0% silver zeolite, no silver in the coated panel and no detectable surface silver. Test panel 2 with 1% silver zeolite had 0.025% silver in the coating, 8.2 mg/L of which was surface silver. Test panels 3 through 6 all had 3% silver zeolite, therefore 0.075% silver in the powdered coating. However, the surface silver concentrations were variable for these panels with panels 3, 4, 5 and 6 having 8.1, 8.6, 4.5, and 15 mg/L surface silver respectively.

Regardless of the measured concentration of surface silver, none of the test panels powder coated with a urethane-cured polyester composition supported bacterial growth as indicated in Table 1. However, the control aluminum panel, panel 1 of Table 1, lacking silver zeolite hence lacking silver, did support bacterial growth. Thus, in urethane-cured polyester powder coating compositions silver zeolite is an effective carrier to supply anti-microbial silver in the powder coating.

TABLE 1
Urethane-Cured Polyester Compositions

| | Test Panels | | | | | |
|---|---|---|---|---|---|---|
| Components | 1 | 2 | 3 | 4 | 5 | 6 |
| Hydroxyl Polyester Resin (parts) | 100 | 100 | 100 | 100 | 100 | 100 |
| Ruco 102 HYD from Rucco Polymer Corp. | | | | | | |
| Hicksville, NY 11801 | | | | | | |
| Caprolactam-Blocked Isocyanate Curing Agent | 22.1 | 22.1 | 22.1 | 22.1 | 22.1 | — |
| (parts) | | | | | | |
| Alcure 4400 from McWhorter Tech. Inc., | | | | | | |
| Ennis, TX 75119 | | | | | | |
| Uretidione-Blocked Isocyanate Curing | — | — | — | — | — | 27.7 |
| Agent (parts) | | | | | | |
| Alcure 4147 from McWhorter Tech. Inc. | | | | | | |
| Flow Aid (parts) | 0.62 | 0.62 | 0.62 | 0.62 | 0.62 | 0.62 |
| L-7605 Silwet Flow Aid from OSI Specialties | | | | | | |
| Degassing Aid (Parts) | 0.60 | 0.60 | 0.60 | 0.60 | 0.60 | 0.60 |
| Benzoin Degassing Aid from Estron Chem. | | | | | | |
| Inc., Calvert City, KY 42029 | | | | | | |
| Carbon Black Pigment (parts) | — | — | — | 1.33 | — | — |
| Raven 1200 Carbon Black Pigment from | | | | | | |
| Columbian Chemicals Pigment Co., Marietta, | | | | | | |
| GA 30062 | | | | | | |
| TiO₂ Pigment (parts) | — | — | — | — | 55.6 | — |
| R-960 from DuPont | | | | | | |
| Filler (parts) | — | — | — | 13 | 13 | — |
| Sparwhite BaSO₄ Filler from Mountain | | | | | | |
| Minerals, Calgary, Alberta, Canada T2P 2Z2 | | | | | | |
| Silver Zeolite | — | 1 | 3 | 3 | 3 | 3 |
| (%, based on the sum of the other components) | | | | | | |
| AJ10D Antimicrobial from AgION Tech. | | | | | | |
| L.L.C. , Wakefield, MA 01880 | | | | | | |
| Silver Concentration in Coating (%) | 0.0 | 0.025 | 0.075 | 0.075 | 0.075 | 0.075 |
| Surface Silver (mg/L) | 0.0 | 8.2 | 8.1 | 8.6 | 4.5 | 15 |
| Bacteriostatic Activity (Kill Efficiency, %) | 0.0 | 99.99 | 99.99 | 99.99 | 99.99 | 99.99 |

Referring to Table 2, aluminum test panels were coated with a carboxyl polyester resin based powder coating composition that was cured with a TGIC curing agent. Test 5 panels 7, 8, 9 and 10 contained 0, 1, 4 and 10 percent silver zeolite, respectively. For test panels 7 through 10, coating compositions also contained a flow aid, a degassing aid, a pigment and a filler.

Test panel 7, shown in Table 2, did not contain silver zeolite in the powder coating composition, thus, there was no silver in the powder coating and no detectable surface silver. Silver zeolite was added to the coating compositions of test panels 8, 9 and 10 giving the powder coated substrates silver concentrations of 0.025%, 0.1% and 0.25% respectively. Detectable surface silver for test panels 8, 9 and 10 were 1.9, 14.8 and 34.4 mg/L respectively.

Unlike the urethane-cured polyester coated panels, the TGIC epoxy-cured polyester coated test panels only showed anti-microbial activity at the highest concentration of zeolite used. That is, test panel ten showed a 98.6% inhibition of bacterial growth as compared to a zeolite-free control whereas test panels 8 and 9 did not exhibit any inhibition of bacterial growth. Thus, in TGIC epoxy-cured polyester powder coating compositions, a higher percentage of silver zeolite may be needed to provide surface silver ions in a concentration that is effective as an anti-microbial agent.

TABLE 2
TGIC Epoxy-Cured Polyester Composition

| | Test Panels | | | |
|---|---|---|---|---|
| Component | 7 | 8 | 9 | 10 |
| Carboxyl Polyester Resin (parts) | 100 | 100 | 100 | 100 |
| Uralac P-2400 from DSM Resins U.S., Inc. | | | | |
| Augusta, GA 30903 | | | | |
| TGIC Curing Agent (parts) | 7.5 | 7.5 | 7.5 | 7.5 |
| Araldite PT-810 from Vantico Inc., | | | | |
| Los Angeles, CA 90023 | | | | |
| Flow Aid (parts) | 1.3 | 1.3 | 1.3 | 1.3 |
| Modaflow III Flow Aid From Estron Chem. | | | | |
| Inc., Calvert City, KY 42029 | | | | |
| Degassing Aid (parts) | 1.2 | 1.2 | 1.2 | 1.2 |
| Benzoin Degassing Aid from Estron Chem. | | | | |
| Inc. | | | | |
| Pigment (parts) | 63 | 63 | 63 | 63 |
| R-960 TiO₂ Pigment from DuPont | | | | |
| Filler (parts) | 14 | 14 | 14 | 14 |
| Sparwhite BaSO₄ Filler from Mountain | | | | |
| Minerals, Calgary, Alberta, Canada T2P 2Z2 | | | | |
| Silver Zeolite | 0 | 1 | 4 | 10 |
| (%, based on the sum of the other | | | | |
| components) AJ10D Antimicrobial from | | | | |
| AgION Tech. L.L.C., | | | | |
| Wakefield, MA 01880 | | | | |
| Silver Concentration in Coating (%) | 0.0 | 0.025 | 0.10 | 0.25 |
| Surface Silver (mg/L) | 0.0 | 1.9 | 14.8 | 34.4 |
| Bacteriostatic Activity (Kill Efficiency, %) | 0.0 | 0.0 | 0.0 | 98.6 |

In sum, both types of powder coating compositions containing a silver zeolite showed anti-microbial activity. However, the urethane-cured powdered coating composition had superior anti-microbial activity.

### EXAMPLE 6

Experiments were performed to determine the anti-microbial effects of silver when a silver zeolite is impact fused to powder coating particles. The silver zeolite, AJ10D containing 2.5% silver as Ag(I), was obtained from AgION™ Technologies, L.L.C., Wakefield, Mass. 01880.

Triglycidylisocyanurate (TGIC)-cured carboxyl-functional polyester coatings were prepared by combining and bag-blending 100 parts of a carboxyl polyester resin (Crylcoat 7309 made by UCB Chemicals Corp., Smyrna, Ga. 30080) and 8 parts of a TGIC curing agent (Araldite PT-810 made by Vantico Inc., Los Angeles, Calif. 90023), as shown in Table 3. In addition to the listed ingredients, small amounts of catalysts, flow aids, degassing aids and toner pigments that are typical of triglycidylisocyanurate (TGIC)-cured powder coatings were also added to the coating mixtures. For example, see Table 2 of Example 5. The silver zeolite was not combined with the other powder coating components at this stage in formulations 1 through 3 of Table 4. However, the silver zeolite AgION™ AJ10D was added to the powder coating premix in formulations 4, 5 and 6 of Table 4. The respective concentrations of zeolite in formulations 4, 5 and 6 were 8, 10 and 12% by weight of the total powder coating composition. See Table 4.

TABLE 3
Coating Composition

| Component | Parts by Weight |
|---|---|
| Carboxyl Polyester Resin | 100 |
| Crylcoat 7309 from UCB Chemicals Corp. | |
| Smyrna, GA 30080 | |
| TGIC Curing Agent | 8 |
| Araldite PT-810 from Vantico Inc., | |
| Los Angeles, CA 90023 | |
| Pigment | 63 |
| R-960 TiO₂ Pigment from DuPont | |

The powder coating components for formulations 1 through 6 were then melt blended through an extruder, the extrudate was solidified between chilled rolls, broken up and ground into a powder to yield powder coating particles. The powders were scalped at 80 mesh to remove coarse particles.

Formulations 1 through 3 were further processed to fuse the silver zeolite to the scalped powder particles by impact fusion as follows. The silver containing zeolite AgION™ AJ10D was added to the preformed powder coating compositions to yield mixtures containing 1, 3 and 5% zeolite by weight for formulations 1, 2 and 3 respectively. For example, to make formulation 1, 0.0505 pounds of AgION™ AJ10D was added to 5.0 pounds of power coating powder, thus, the resultant mixture contained 1% AJ10D zeolite.

The powder coating mixtures for formulations 1 through 3 were then blended at 3000 rpm in a 10-liter capacity Papenmeier TGHK-10 High Intensity Mixer (Merlin Process Equipment, Inc., Houston, Tex.). During blending the temperature of the mixtures rose to 57° C. This temperature is approximately equivalent to the glass transition temperature of the powder. To achieve 57° C., blend times for the mixtures of formulations 1, 2 and 3 were adjusted accordingly. The high intensity blending process homogeneously dispersed the silver zeolite and substantially entirely fused it to the surface of the powder coating particles. After blending, the powder coating formulations 1, 2 and 3 were cooled and sieved through a 60 mesh screen to break up lumps produced during the impact fusion process.

To test for anti-microbial effect, three 2 inch by 2 inch aluminum panels were coated with each formulation (1 through 6) using an electrostatic spray gun. The panels were then baked for 10 minutes at 400 F (204 C) and cooled. The panels were then inoculated with suspensions of Escherichia coli and incubated at 37° C. for 24 hours. The panels were then rinsed and the rinsate was serially diluted. The serial dilutions were applied to agar medium and further incubated at 37° C. The agar medium was inspected to determine the percentage of bacterial growth as compared to a control. The results appear in Table 4 as “Kill Efficiency”.

TABLE 4
Bacterial Kill Efficiency and Incorporation Method

| | AJ10D | | |
|---|---|---|---|
| | (Silver containing | | Kill |
| | zeolite) | Kill Efficiency (%) | Efficiency (%) |
| Formulation | Loading (Wt. %) | Bonded AJ10D | AJ10D in Premix |
| 1 | 1 | 99.99 | Not Tested |
| 2 | 3 | 99.99 | Not Tested |
| 3 | 5 | 99.99 | Not Tested |
| 4 | 8 | Not Tested | 0 |
| 5 | 10 | Not Tested | 99.99 |
| 6 | 12 | Not Tested | 99.99 |

The results of Table 4 indicate that panels coated with TGIC-cured powder coating containing silver zeolite generally do not support bacterial growth. Those panels containing impact fused zeolite were able to inhibit bacterial growth at a lower concentration of zeolite than those panels containing silver zeolite that had been premixed, bag-blended and then extruded. For example, formulation 1 containing 1% impact fused AJ10D was able to inhibit 99.99% of bacterial growth. In contrast, of those formulations that contained premixed AJ10D, formulation 5 containing 10% zeolite was the lowest concentration of premixed silver zeolite to inhibit bacterial growth.

Although both impact fusion and premixing produce a homogeneous distribution of the silver zeolite, the impact fused zeolite may be substantially entirely present on the surface of the powder particles rather than being distributed through the bulk of the powder particles. When positioned at the surface, the silver zeolite may be preferentially present on the surface of the film that forms when the powder melts during curing. As a result, the silver zeolite may be better positioned on a coated substrate to produce anti-microbial effects. Thus, for some applications, impact fused silver zeolite may provide anti-microbial effects at lower concentrations than premixed silver zeolite.

### EXAMPLE 7

Experiments were performed to determine the anti-microbial effects of silver in a typical urethane powder coating when a zirconium phosphate acts as a silver carrier. Silver zirconium phosphate (AlphaSan RC 5000 containing 3.8% silver) was obtained from Milliken Chemical, Spartanburg, S.C. 29304.

Typical urethane powder coatings were prepared by combining and blending 100 parts carboxyl polyester resin (Rucote 102 from Ruco Polymers, Hicksville, N.Y. 11801), 25.4 parts blocked isocyanate curing agent (Vestagon B-1530 from Creanova, Inc., Somerset, N.J. 08873), 55.3 parts pigment (TR-92 TiO₂ pigment from Huntsman Tioxide, Downers Grove, Ill. 60515) and 13.2 parts filler (Sparwhite BaSO₄ from Mountain Minerals, Calgary, Alberta, Canada T2P 2Z2), as shown in Table 5. In addition to the listed ingredients, small amounts of flow aids and degassing aids (less than 2% each) were added to the powder coating formulations.

Three different powder coating formulations were prepared by combining and blending silver zirconium phosphate with the above listed components of the urethane based powder coating. For example, referring to Table 5, formulation 2 contained 1% by weight based on the sum of the other powder coating components, and formulation 3 contained 2% by weight of the silver zirconium phosphate. Formulation 1, a control formulation, did not contain silver zirconium phosphate; therefore, silver was not present in this powder coating composition. In contrast, formulations 2 and 3 contained 0.038% and 0.076% respectively, silver in the powder coating composition (the concentration of silver in AlphaSan RC 5000 (3.8%) multiplied by the % weight of AlphaSan added to the particular formulation), as shown in Table 5.

Once blended, all three powder coating formulations listed in Table 5 were melt-extruded, solidified between chilled rolls, and broken up and ground into a powder as previously described.

TABLE 5
Silver Zirconium Phosphate in Urethane Coatings

| | Formulations | | |
|---|---|---|---|
| Component | 1 | 2 | 3 |
| Carboxyl Polyester Resin (parts) | 100 | 100 | 100 |
| Rucote 102 from Ruco Polymers, Hicksville, | | | |
| NY 11801 | | | |
| Blocked Isocyanate Curing Agent (parts) | 25.4 | 25.4 | 25.4 |
| Vestagon B-1530 from Creanova, Inc., | | | |
| Somerset, NJ 08873 | | | |
| Pigment (parts) | 55.3 | 55.3 | 55.3 |
| TR-92 TiO₂ Pigment from Huntsman Tioxide, | | | |
| Downers Grove, IL 60515 | | | |
| Filler (parts) | 13.2 | 13.2 | 13.2 |
| Sparwhite BaSO₄ from Mountain Minerals, | | | |
| Calgary, Alberta, Canada T2P 2Z2 | | | |
| Silver Zirconium Phosphate | 0 | 1 | 2 |
| (%, based on the sum of the other components) | | | |
| AlphaSan RC 5000 containing 3.8% silver | | | |
| from Milliken Chemical, Spartanburg, SC | | | |
| 29304 | | | |
| Silver Concentration in Coating (%) | 0.0 | 0.038 | 0.076 |
| Bactericidal Activity (Kill Efficiency, %) | 0 | 99.99 | 99.99 |

Note:
Each formulation also contained minor amounts (<2% each) of flow aids and degassing aids.

Testing for anti-microbial activity was carried out as described in Examples 5 and 6. Generally, the powder coatings were sprayed onto test panels using electrostatic spray equipment and then they were thermally cured at 400° F. for 10 minutes in a convection-heated oven.

Anti-microbial efficacy was also tested as described in Examples 5 and 6. For example, test panels were exposed to bacteria, incubated and rinsed and the rinsate was serially diluted. The serial dilutions were then applied to agar, further incubated and then inspected to determine bacterial growth. The results appear in Table 5 as Bactericidal Activity (Kill Efficiency, %).

The results of these experiments indicate that silver zirconium phosphate is an effective anti-microbial agent when combined with urethane based powder coating components at the premix phase. As expected, formulation 1, which did not contain silver zirconium phosphate, did not exhibit bactericidal activity. In other words, formulation 1 did not inhibit bacterial growth. However, formulations 2 and 3 both exhibited bactericidal activity at a 99.99% Kill Efficiency. That is, formulations 2 and 3 containing 1% and 2% respectively of the AlphaSan RC 5000 killed 99.99% of the bacterial growth. Thus, silver, in the form of silver zirconium phosphate is an effective anti-microbial agent when incorporated into urethane powder coatings at the premix stage.

### EXAMPLE 8

Experiments were performed to determine the anti-microbial effects of silver in an epoxy powder coating when silver, carried by a zirconium phosphate, is either added to the powder coating components during the premix phase or impact fused to the scalped powder coating particles or both. Silver zirconium phosphate (AlphaSan RC 5000 containing 3.8% silver) was obtained from Milliken Chemical, Spartanburg, S.C. 29304.

Epoxy powder coatings were prepared by combining and blending 84 parts epoxy resin (DER 642U from The Dow Chemical Co., Midland, Mich. 48674), 16 parts epoxy resin (DER 672U from The Dow Chemical Co., Midland, Mich. 48674), 29 parts curing agent (DEH 84 from The Dow Chemical Co., Midland, Mich. 48674), 15 parts curing agent (DEH 85 from The Dow Chemical Co., Midland, Mich. 48674), 73 parts pigment (TR-92 TiO₂ pigment from Huntsman Tioxide, Downers Grove, Ill. 60515), as shown in Table 6. In addition to the listed components, each powder coating composition contained small amounts (less than 2% each) of waxes, flow aids and degassing aids.

The above listed powder coatings components were processed as previously described by the melt extrusion method. Generally, the components were blended, melt extruded, cooled, broken up and ground into fine powder coating particles, which were then scalped to remove any coarse particles.

Referring to Table 6, formulation 1 served as a control and did not have any silver zirconium phosphate added to the other powder coating components. Formulation 2 contained 1% by weight, based on the sum of the other components in the powder coating, of the silver zirconium phosphate. In this case, the silver zirconium phosphate was combined and blended with the other powder coating components for melt extrusion. Formulation 3 also contained 1% by weight, based on the sum of the other components, of the silver zirconium phosphate. However, in this case, instead of blending with the other components prior to melt extrusion, the anti-microbial agent was impact fused to the scalped powder coating particles. Impact fusion was performed as described in Example 6. Formulation 4 of Table 6 contained 2% by weight, based on the sum of the other components, of the silver zirconium phosphate. In this case, 1% was blended with the other powder coating components during the premix phase of the melt extrusion method and 1% was impact fused to scalped powder coating particles.

The total concentration of silver in powder coating formulations 1 through 4 was 0, 0.038%, 0.038% and 0.076% respectively. For example, formulation 3 had 1% by weight AlphaSan RC 5000 wherein the AlphaSan contains 3.8% silver. Thus, the total silver concentration, by percent, in formulation 3 is 0.038%.

TABLE 6
Silver zirconium phosphate in Epoxy Coatings

| | Formulations | | | |
|---|---|---|---|---|
| Component | 1 | 2 | 3 | 4 |
| Epoxy Resin (parts) | 84 | 84 | 84 | 84 |
| DER 642U from The | | | | |
| Dow Chemical Co., | | | | |
| Midland, MI 48674 | | | | |
| Epoxy Resin (parts) | 16 | 16 | 16 | 16 |
| DER 672U from The | | | | |
| Dow Chemical Co., | | | | |
| Midland, MI 48674 | | | | |
| Curing Agent (parts) | 29 | 29 | 29 | 29 |
| DEH 84 from The Dow | | | | |
| Chemical Co., Midland, | | | | |
| MI 48674 | | | | |
| Curing Agent (parts) | 15 | 15 | 15 | 15 |
| DEH 85 from The Dow | | | | |
| Chemical Co., Midland, | | | | |
| MI 48674 | | | | |
| Pigment (parts) | 73 | 73 | 73 | 73 |
| TR-92 TiO₂ Pigment | | | | |
| from Huntsman Tioxide, | | | | |
| Downers Grove, IL | | | | |
| 60515 | | | | |
| Silver zirconium phos- | 0 | 1 | 1 | 2 |
| phate (%, based on the | | extruded | impact | 1% impact fused |
| sum of the other compo- | | | fused | 1% extruded |
| nents) | | | | |
| AlphaSan RC 5000 con- | | | | |
| taining 3.8% silver from | | | | |
| Milliken Chemical, | | | | |
| Spartanburg, SC 29304 | | | | |
| Silver Concentration in | 0.0 | 0.038 | 0.038 | 0.076 |
| Coating (%) | | | | |
| Bactericidal Activity | 0 | 0 | 99.99 | 99.99 |
| (Kill Efficiency, %) | | | | |

Note:
Each formulation also contained minor amounts (<2% each) of waxes, flow aids and degassing aids.

Formulations 1 through 4 were tested for anti-microbial activity as previously described in Examples 5 and 6. Generally, the powder coatings were sprayed onto test panels using electrostatic spray equipment and then they were thermally cured at 400° F. for 10 minutes in a convection-heated oven.

Anti-microbial efficacy was also tested as described in Examples 5 and 6. For example, test panels were exposed to bacteria, incubated, rinsed and the rinsate was serially diluted. The serial dilutions were then applied to agar, further incubated and then inspected to determine bacterial growth. The results appear in Table 6 as Bactericidal Activity (Kill Efficiency, %).

Referring to Table 6, as expected, formulation 1 supported bacterial growth due to the absence of an anti-microbial agent. Formulation 2, containing 1% by weight of the silver zirconium phosphate, also supported bacterial growth. In other words, formulation 2 did not exhibit bactericidal activity. In contrast, neither formulation 3 nor formulation 4 supported bacterial growth, each exhibiting 99.99% kill efficiency.

Although both formulations 2 and 3 both contained 1% by weight of the silver zirconium phosphate, the preparation of these formulations differed in that the anti-microbial agent was combined with the other powder coating components during the premix phase in formulation 2 whereas it was impact fused to scalped powder coating particles in formulation 3. Thus, low levels of silver zirconium phosphate, such as 1%, may serve as an effective anti-microbial agent when it is impact fused to but not pre-mixed in epoxy powder coatings.

Formulation 4 combined the two methods of dispersing silver zirconium phosphate in a powder coating composition. That is, in formulation 4 silver zirconium phosphate was added to the premix and it was impact fused to scalped power particles. As a result, formulation 4 exhibited 99.99% kill efficiency. Thus, dual route incorporation of the silver zirconium phosphate exhibited a kill efficiency that is the same as the kill efficiency observed with the single route of incorporation of impact fusion. However, because impact fusion tends to substantially localize the silver in higher concentrations at the surface of the substrate, dual route incorporation of the silver zirconium phosphate may be desirable when the coating will be subject to environments that may remove the outer layer of the powder coating. Thus, dual route incorporation of the silver zirconium phosphate may maintain the silver levels, hence the anti-microbial activity, when abrasion removes surface silver.

### EXAMPLE 9

Experiments are performed to determine the effects of Ultraviolet (UV)-curing on the anti-microbial effects of silver zeolite. The silver zeolites, AK10D, containing 5.0% silver as Ag(I), and AJ10D, containing 2.5% silver as Ag(I), were obtained from AgION™ Technologies, L.L.C., Wakefield, Mass. 01880.

Ultraviolet-cured anti-microbial coatings are prepared by combining and blending 100 parts unsaturated polyester resin (Uvecoat 3002 from UCB Chemicals Corp., 1620 Drogenbos, Belgium), 2.5 parts cure initiator (IRGACURE® 2959 from Ciba® Specialty Chemicals, Tarrytown, N.Y. 10591) as shown Table 7. In addition to the listed components, the powder coating compositions listed in Table 7 each contain small amounts (less than 2% each) of flow aids and degassing aids.

Referring to Table 7, four different UV-cured powder coating formulations are prepared. Formulation 3 has 1% by weight, based on the sum of the other components, of the silver zeolite AK1OD. In this formulation, the silver zeolite is blended with the other powder coating components during the premix phase before the melt extrusion of the method described above.

Formulation 2 contains 1% by weight, based on the sum of the other components, of the silver zeolite AK10D. Formulation 4 contains 2% by weight, based on the sum of the other components, of the silver zeolite AJ10D. However, in this case, instead of blending with the other components, the silver zeolites of formulation 2 and formulation 4 are impact fused to scalped powder coating particles. Impact fusion is performed as described in Example 6. Formulation 1 serves as a control, thus, silver zeolite is not added to this formulation.

The total silver concentration for formulations 1 through 4 are 0.0%, 0.05%, 0.05% and 0.05% respectively. For example, formulation 3 contains 1% AK1OD having 5.0% silver. Thus, the total silver concentration, by percent, in formulation 3 is 0.05 percent.

TABLE 7
Ultraviolet Light-Cured Antimicrobial Coatings

| | Formulations | | | |
|---|---|---|---|---|
| Component | 1 | 2 | 3 | 4 |
| Unsaturated Polyester Resin | 100 | 100 | 100 | 100 |
| (parts), | | | | |
| Uvecoat 3002 from UCB | | | | |
| Chemicals Corp., 1620 | | | | |
| Drogenbos, Belgium | | | | |
| Cure Initiator (parts), Irgacure | 2.5 | 2.5 | 2.5 | 2.5 |
| 2959 from Ciba Specialty | | | | |
| Chemicals, Tarrytown, NY | | | | |
| 10591 | | | | |
| Silver Zeolite | 0 | 1.0 | 1.0 | 0 |
| (%, based on the sum of the | | impact fused | extruded | |
| other components) | | | | |
| AgION AK10D containing | | | | |
| 5.0% silver as Ag(I) from | | | | |
| AgION Technologies, L.L.C. | | | | |
| Wakefield, MA 01880 | | | | |
| Silver Zeolite | 0 | 0 | 0 | 2.0 |
| (%, based on the sum of the | | | | impact |
| other components) | | | | fused |
| AgION AJ10D containing | | | | |
| 2.5% silver as Ag(I) from | | | | |
| AgION Technologies, L.L.C. | | | | |
| Wakefield,MA 01880 | | | | |
| Silver Concentration in | 0.0 | 0.05 | 0.05 | 0.05 |
| Coating (%) | | | | |
| Bactericidal Activity (Kill | 0 | 99.99 | 0 | 99.99 |
| Efficiency, %) | | | | |

Note:
Each formulation also contains minor amounts (<2% each) of flow aids and degassing aids.

The anti-microbial powder coating particles are sprayed onto substrates such as 2 inches×2 inches aluminum panels using electrostatic spray equipment. The coatings are then fused to the substrate by a combination of infrared and convection heating so that the surface temperature reaches about 250° F. Curing takes place by exposing the coated and fused substrates to between 400 and 600 Joules/centimeter of UV radiation produced by H-lamps manufactured by Fusion UV Systems, Inc., Gaithersburg, Md.

Anti-microbial activity is tested as described in Examples 5 and 6. For example, the panels are exposed to bacteria, incubated, then rinsed and the rinsate is serially diluted. Serial dilutions are applied to agar, further incubated and inspected to determine bacterial growth. The results appear in Table 7 as Bactericidal Activity (Kill Efficiency, %).

Referring to Table 7, formulation 1 supports bacterial growth due to the absence of silver zeolite. However, formulation 2, with 1% impact fused silver zeolite having a higher percentage of silver, demonstrates bactericidal activity at a 99.99% kill efficiency. In contrast, formulation 3, also containing 1% silver zeolite also having a higher percentage of silver, which is added during the premix phase, does not show any bactericidal activity. That is, formulation 3 supports bacterial growth.

Formulation 4, with 2% impact fused silver zeolite having a lower percentage of silver also shows bactericidal activity at a 99.99% kill efficiency. Thus, in powder coating compositions that are based on ultraviolet radiation cured unsaturated polyester resins, impact fused silver zeolite demonstrates bactericidal activity at low concentrations.

### EXAMPLE 10

Experiments were performed to determine whether certain additives, such as pigments, affect the efficacy of anti-microbial agents, such as silver zeolite. The silver zeolite, AJ10D containing 2.5% silver as Ag(I), was obtained from AgION™ Technologies, L.L.C., Wakefield, Mass. 01880.

Triglycidylisocyanurate (TGIC)-cured carboxyl-functional polyester coatings were prepared by combining and bag-blending 100 parts of a carboxyl polyester resin (Crylcoat 7309 made by UCB Chemicals Corp., Smyrna, Ga. 30080) and 8 parts of a TGIC curing agent (Araldite PT-810 made by Vantico Inc., Los Angeles, Calif. 90023), as described in Table 3 of Example 6. However, the pigment in these experiments is different from the pigment listed in Table 3. As shown in Table 8, 63 parts of the pigment R-706 TiO₂ (DuPont Chemical Corp., Wilmington, Del.) was added to formulations 8a through 8e whereas 63 parts of the pigment TR-92 TiO₂ (Huntsmen Tioxide, Downers Grove, Ill. 60515) was added to formulations 8-aa, 8-bb and 8-cc. The R-706 TiO₂ pigment is known to contain 300-600 parts per million (ppm) chloride ion. Chloride ions are largely present in silica and alumina surface treatments and have precipitated onto the TiO₂ pigment particles during their manufacture. In contrast, the TR-92 pigment contains less than 10 ppm chloride ion. For all of the formulations in this experiment the silver zeolite was blended with the other powder coating components during the premix phase before the melt extrusion of the method as described in the specification.

Formulations 8-a and 8-aa contained 3% of the silver zeolite. Formulations 8-b and 8-bb contained 5% of the silver zeolite, whereas formulations 8-c and 8-cc contained 8% of the silver zeolite. Lastly, formulation 8-d contained 10% of the silver zeolite and formulation 8-e contained 12% silver zeolite. The percentage of silver zeolite added to these powder coating formulations is based on the weight of the silver zeolite added as compared to the sum of the weight of the other components.

After combining and bag blending, the powder coating components were melt-extruded as previously described. The extrudate was solidified by chilling between rolls, broken up and ground into a powder to yield powder coating particles. The powder coating particles were then scalped at 80 mesh to remove coarse particles.

Testing for anti-microbial activity was carried out as described in Examples 5 and 6. Generally, the powder coatings were sprayed onto test panels using electrostatic spray equipment and then they were thermally cured at 400° F. for 10 minutes in a convection-heated oven.

Anti-microbial efficacy was also tested as described in Examples 5 and 6. For example, test panels were exposed to bacteria, incubated, rinsed and the rinsate was serially diluted. The serial dilutions were then applied to agar, further incubated and inspected to determine bacterial growth. The results appear in Table 8 as a percent of the Kill Efficiency.

TABLE 8
Bacterial Kill Efficiency and Pigment Chloride

| AJ10D | R-706 Pigmented | | TR-92 Pigmented | |
|---|---|---|---|---|
| (Silver containing | 63 parts | | 63 parts | |
| zeolite) | Formula- | Kill | Formula- | Kill |
| Loading (Wt. %) | tion | Efficiency % | tion | Efficiency % |

| | | | | |
|---|---|---|---|---|
| 3 | 8-a | 0 | 8-aa | 99.99 |
| 5 | 8-b | 0 | 8-bb | 99.99 |
| 8 | 8-c | 0 | 8-cc | 99.99 |
| 10 | 8-d | 99.99 | — | — |
| 12 | 8-e | 99.99 | — | — |

Referring to Table 8, those formulations prepared with the R-706 pigment do not display any bacterial Kill Efficiency until the silver zeolite is present at a concentration that is at least 10% by weight of the powder coating composition. In contrast, the formulations containing the TR-92 pigment show 99.99% bacterial Kill Efficiency at concentrations as low as 3% by weight of the total powder coating composition. Because R-706 is known to have a high chloride ion concentration and TR-92 is known to have a low chloride ion concentration, these results suggest that the replacement of R-706 with TR-92 improves the anti-microbial kill efficiency by reducing the concentration of chloride ions in the powder coating composition. Conceivably, with TR-92, the ability of silver to migrate through the coating is improved and/or the solubility of the silver is increased due to the diminished concentration chloride ions in the powder coating to bind silver and precipitate it out of the powder coating composition as silver chloride.

Chloride is known to react with silver to form silver chloride. Thus, the silver in the powder coating formulations with a high chloride ion concentration may be more readily precipitated out as silver chloride thereby reducing the availability of the silver to migrate through the powder coating.

What is claimed is:

1. An anti-microbial powder coating composition comprising one or more anti-microbial agents impact fused to particles of a resin-based powder.

2. The composition of claim 1 wherein the anti-microbial agent is substantially entirely impact fused to the surface of the resin-based powder particles.

3. The composition of claim 2 wherein the anti-microbial agent is an anti-microbial metal or metal ion.

4. The composition of claim 3 wherein the anti-microbial metal or metal ion is silver.

5. The composition of claim 4 wherein the silver is in the form of a silver ion carried by a zeolite.

6. The composition of claim 4 wherein the silver is supplied by a silver salt.

7. The composition of claim 4 wherein the silver is supplied by a silver zirconium phosphate.

8. The composition of claim 4 wherein the silver is supplied by an organic composition containing silver.

9. The composition of claim 5 wherein the powder coating composition comprises a thermosetting composition based on a cured polyester resin composition.

10. The composition of claim 9 wherein the polyester resin composition is cured with a triglycidylisocyanurate.

11. The composition of claim 10 wherein the silver containing zeolite is about 1 to 5 percent by weight of the sum of the components comprising the powder coating composition.

12. The composition of claim 7 wherein the powder coating composition comprises a thermosetting composition based on a cured epoxy resin composition.

13. The composition of claim 12 wherein the silver zirconium phosphate is about 1 percent by weight of the sum of the components comprising the powder coating composition.

14. The composition of claim 5 wherein the powder coating composition comprises a radiation curable composition based on an ultraviolet curable polyester resin.

15. The composition of claim 14 wherein the ultraviolet curable polyester resin is an unsaturated polyester resin.

16. The composition of claim 14 wherein the silver zeolite is about 1 to 2 percent by weight of the sum of the components comprising the powder coating composition.

17. The composition of claim 1 including one or more anti-microbial metal or metal ions homogeneously dispersed within particles of the powder coating composition.

18. The composition of claim 17 wherein the homogeneously dispersed anti-microbial metal or metal ion is silver.

19. The composition of claim 18 wherein the homogeneously dispersed silver is in the form of a silver zirconium phosphate.

20. The composition of claim 18 wherein the homogeneously dispersed silver is in the form of a silver ion carried by a zeolite.

21. The composition of claim 19 wherein the homogeneously dispersed silver zirconium phosphate is about 1 percent by weight of the sum of the components comprising the powder coating composition.

22. The composition of claim 20 wherein the homogeneously dispersed silver zeolite is about 1 to 5 percent by weight of the powder coating composition.

23. A method for preparing an anti-microbial powder coating composition comprising impact fusing one or more anti-microbial agents to particles of a resin-based powder.

24. The method of claim 23 further including absent the anti-microbial agent, blending the components of the powder coating composition using a premixer, feeding the mixture into an extruder, heating the mixture to a temperature high enough to melt it, cooling the melt, and processing the solid extrudate into a coating powder.

25. The method of claim 24 wherein impact fusing includes impact fusing the anti-microbial agent to the coating powder so that the anti-microbial agent is substantially entirely fused to the surface of the coating powder particles.

26. The method of claim 24 wherein impact fusing the anti-microbial agent includes mixing the anti-microbial agent with the coating powder, blending the mixture in a high intensity mixer, cooling the mixture and processing the mixture into an anti-microbial coating powder.

27. The method of claim 26 further including adjusting the blending time to approximately achieve the glass transition temperature.

28. A method for preparing an anti-microbial powder coating composition comprising blending the components of the powder coating composition using a premixer, feeding the mixture into an extruder, heating the mixture to a temperature high enough to melt it, cooling the melt, processing the solid extrudate into a coating powder, and impact fusing one or more anti-microbial agents to the particles of the coating powder.

29. The method of claim 28 wherein impact fusing one or more anti-microbial agents to the particles of the coating powder includes mixing the anti-microbial agent with the coating powder, blending the mixture in a high intensity mixer, cooling the mixture and processing the mixture into an anti-microbial coating powder.

30. The method of claim 29 further including adjusting the blending time to approximately achieve the glass transition temperature.

31. An anti-microbial powder coating composition comprising an anti-microbial silver zirconium phosphate that is homogeneously dispersed within particles of a resin-based powder.

32. The composition of claim 31 wherein the powder coating composition comprises a thermosetting composition based on a cured polyester resin composition.

33. The composition of claim 32 wherein the polyester resin composition is cured with a urethane curing agent.

34. The composition of claim 33 wherein the silver zirconium phosphate is about 1 percent to 2 percent of the sum of the components comprising the powder coating composition.

35. An anti-microbial powder coating composition comprising one or more anti-microbial metals or metal ions homogeneously dispersed within particles of a radiation curable resin-based powder.

36. The composition of claim 35 wherein the anti-microbial metal or metal ion is silver.

37. The composition of claim 36 wherein the silver is a silver ion carried by a zeolite.

38. The composition of claim 36 wherein the resin-based powder is ultraviolet-radiation curable.

39. The composition of claim 38 wherein the resin is a polyester resin.

40. The composition of claim 35 further comprising a cure initiator.

41. The composition of claim 40 wherein the cure initiator is a free radical producing cure initiator.

42. The composition of claim 40 wherein the cure initiator is a cation producing cure initiator.

43. The composition of claim 36 wherein the silver zeolite is about 1 to 2 percent by weight of the sum of the components comprising the powder coating composition.

44. A powder coating composition comprising one or more anti-microbial metals or metal ions homogeneously dispersed within particles of a resin-based powder, said resin-based powder formulated such that the components do not inhibit the migration or decrease the solubility of said anti-microbial metals or metal ions.

45. The powder coating composition of claim 44 wherein the anti-microbial metal or metal ion is silver.

46. The powder coating composition of claim 45 wherein the silver is in the form of a silver ion carried by a zeolite.

47. The powder coating composition of claim 45 wherein the component that inhibits the migration or decreases the solubility of said silver is an ionic halide that is not already associated with silver.

48. The powder coating composition of claim 47 wherein the concentration of the ionic halide in the powder coating composition is less than 300 parts per million.

49. The powder coating composition of claim 47 wherein the concentration of the ionic halide in the powder coating composition is less than 50 parts per million.

50. The powder coating composition of claim 47 wherein the concentration of the ionic halide in the powder coating composition is less than 10 parts per million.

51. The powder coating composition of claim 47 wherein said ionic halide is chloride.

52. The powder coating composition of claim 46 wherein said silver zeolite is about 3 percent to 12 percent by weight of the sum of the components of the powder coating composition.
